(19) United States
(12) Patent Application Publication
(10) Pub. No.: US 2017/0007149 A1
(43) Pub. Date: Jan. 12, 2017

(54) MICROWAVE IMAGING DEVICE AND METHOD

(71) Applicant: Wistron Corporation, New Taipei City (TW)

(72) Inventor: Ssu-Han Ting, New Taipei City (TW)

(21) Appl. No.: 14/872,170

(22) Filed: Oct. 1, 2015

(30) Foreign Application Priority Data
Jul. 9, 2015 (TW) 104122378

(51) Int. Cl. A61B 5/05; A61B 5/00

(52) U.S. Cl. CPC A61B 5/0507; A61B 5/7278; A61B 5/004

(57) ABSTRACT

Microwave imaging device and method are provided, and the microwave imaging device includes a scan circuit, a reception circuit and an image generator. The scan circuit transmits a plurality of electromagnetic waves in a plurality of scan bands toward a target object. The scan bands are respectively corresponding to a plurality of biological tissues in the target object. The reception circuit receives the electromagnetic waves passing through the target object, and generates a plurality of energy values according to the received electromagnetic waves. The image generator looks up a plurality of gray level look-up tables by using the energy values to generate a plurality of gray level values, and generates a detection image corresponding to the biological tissues according to the gray level values.

### CROSS-REFERENCE TO RELATED APPLICATION

This application claims the priority benefit of Taiwan application serial no. 104122378, filed on Jul. 9, 2015. The entirety of the above-mentioned patent application is hereby incorporated by reference herein and made a part of this specification.

### BACKGROUND OF THE INVENTION

Field of the Invention

The invention relates to an imaging technology, and particularly to a microwave imaging device and method.

Description of Related Art

Along with the improvement of medical sciences, the imaging devices which are currently used in medical imaging detection include: X-ray imaging device, digital imaging device, and magnetic resonance imaging device. The detection image generated by the X-ray imaging device has 10%˜20% as false negative. Therefore, the patients often need to be further examined through the digital imaging device or the magnetic resonance imaging device. However, the cost of the hardware of the digital imaging device and the magnetic resonance imaging device is high so the detection fee is also high when the digital imaging device or the magnetic resonance imaging device is applied to clinical diagnosis, and then burden the patient. Therefore, how to apply the modern medical technology to reduce the cost of the hardware of the imaging device so as to reduce the burden of the patient is an indispensable issue in the development of the imaging device.

### SUMMARY OF THE INVENTION

The invention provides a microwave imaging device and method, which can reduce the cost of the hardware of the microwave imaging device so as to reduce the detection fee effectively when the microwave imaging device is applied to clinical diagnosis.

The microwave imaging device in the invention includes a scan circuit, a reception circuit and an image generator. The scan circuit transmits a plurality of electromagnetic waves in a plurality of scan bands toward a target object. The scan bands are respectively corresponding to a plurality of biological tissues in the target object. The reception circuit receives the electromagnetic waves passing through the target object, and generates a plurality of energy values according to the received electromagnetic waves. The image generator looks up a plurality of gray level look-up tables by using the energy values to generate a plurality of gray level values, and generates a detection image corresponding to the biological tissues according to the gray level values.

The microwave imaging method in the invention is adapted to the microwave imaging device, and includes the following steps. Transmitting a plurality of electromagnetic waves in a plurality of scan bands toward a target object through a transmitting antenna array in the microwave imaging device, wherein the scan bands are respectively corresponding to a plurality of biological tissues in the target object. Receiving the electromagnetic waves passing through the target object through a receiving antenna array in the microwave imaging device. Generating a plurality of energy values according to the received electromagnetic waves. Looking up a plurality of gray level look-up tables by using the energy values to generate a plurality of gray level values, and generating a detection image corresponding to the biological tissues according to the gray level values.

Based on the above, in the invention, the electromagnetic waves are used to scan the target object, and the gray level values of the detection image are generated according to the energy values of the electromagnetic waves which pass through the target body. Therefore, the cost of the hardware of the microwave imaging device is reduced so as to reduce the detection fee effectively when the microwave imaging device is applied to clinical diagnosis so that the burden of the patient is effectively reduced.

In order to make the aforementioned and other features and advantages of the invention more comprehensible, embodiments accompanying figures are described in detail below.

### BRIEF DESCRIPTION OF THE DRAWINGS

FIG. 1 is a schematic view of a microwave imaging device according to one embodiment of the present invention.

FIG. 2 is a flowchart illustrating a microwave imaging method according to one embodiment of the present invention.

### DESCRIPTION OF THE EMBODIMENTS

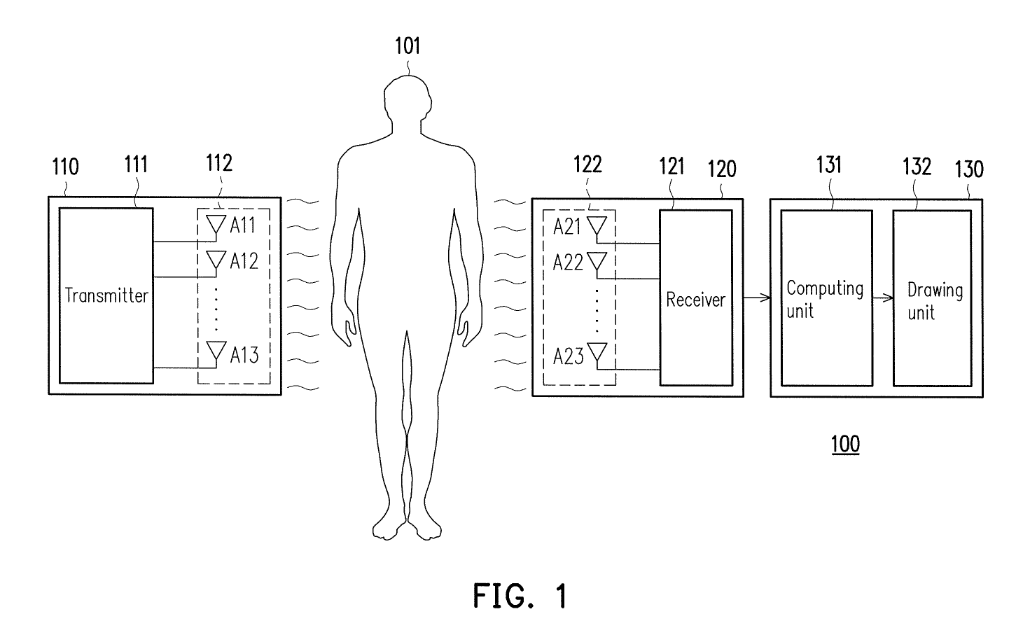

FIG. 1 is a schematic view of a microwave imaging device according to one embodiment of the present invention. As shown in FIG. 1, the microwave imaging device 100 can be used for generating an image of an internal structure of a target object 101 (such as, a human body), so that the doctor can diagnose diseases based on the image. Specifically, the microwave imaging device 100 includes a scan circuit 110, a reception circuit 120, and an image generator 130. In addition, the scan circuit 110 includes a transmitter 111 and a transmitting antenna array 112. The reception circuit 120 includes a receiver 121 and a receiving antenna array 122. The image generator 130 includes a computing unit 131 and a drawing unit 132.

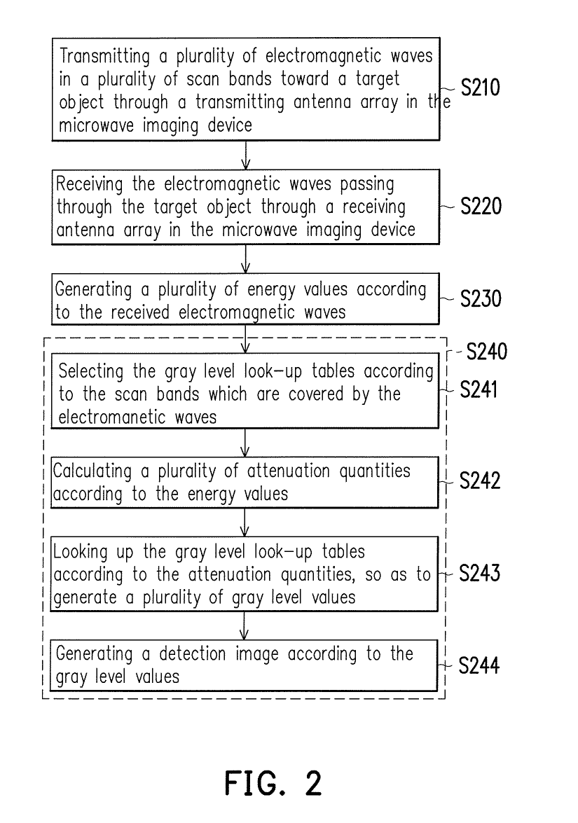

FIG. 2 is a flowchart illustrating a microwave imaging method according to one embodiment of the present invention. Please refer to FIG. 1 and FIG. 2 simultaneously in the following description of the operation of the microwave imaging device. As shown in Step S210, the scan circuit 110 transmits a plurality of electromagnetic waves in a plurality of scan bands toward a target object 101. For example, the transmitter 111 can transmit a plurality of driving signals to the transmitting antenna array 112 to make the transmitting antenna array 112 transmit the electromagnetic waves. In addition, the electromagnetic waves pass through the target object 101 along the propagation path of the transmitting antenna array 112. In other words, the electromagnetic waves enter the target object 101 along the propagation path, and penetrate the biological tissues inside the target object 101.

On the other hand, as shown in Step S220 and Step S230, the reception circuit 120 can receive the electromagnetic waves passing through the target object 101 and can generate a plurality of energy values according to the received electromagnetic waves. For example, the reception circuit 120 can receive the electromagnetic waves passing through the target object 101 through the receiving antenna array 122. Each of the transmitting antenna array 112 and the receiving antenna array 122 includes a plurality of antenna elements. For example, the transmitting antenna array 112 includes the antenna elements A11˜A13, and the receiving antenna array 122 includes the antenna elements A21˜A23. Therefore, the microwave imaging device 100 has a multiple-input multiple-output (MIMO) transmission mechanism. In addition, the receiver 121 can calculate the energy values according to the electromagnetic waves which are received by the receiving antenna array 122. The energy values can be, for example, the received signal-strength indicator (RSSI) values.

It is worth mentioning that the biological tissues of the target object are the conductive medium of the electromagnetic wave, and different biological tissues have different electrical properties. Therefore, the scan circuit 110 can transmit a plurality of electromagnetic waves in a plurality of scan bands, and each of the scan bands is corresponding to a biological tissue, so as to perform scanning for the biological tissues in the target object simultaneously. In addition, when the electromagnetic waves propagate inside the target object, the target object can attenuate the energy of the electromagnetic waves, and the attenuation quantities are related to the electrical properties of the biological tissues of the target object.

Specifically, the electrical properties of the biological tissues of the target object can be defined by the conductivity and the dielectric constant. In addition, the biological tissues have different conductivity characteristics and different dielectric constants according to the electromagnetic waves at different frequencies. Furthermore, considering the same biological tissue, the healthy tissue and the malignant tissue have different conductivity characteristics and different dielectric constants respectively. In other words, the attenuation degrees of the electromagnetic wave according to the healthy tissue and the malignant tissue are not the same so that the malignant tissue in the target object can be detected through the attenuation quantity of the electromagnetic wave.

For example, considering the breast tissue of the human body, the conductivity characteristics and the dielectric constants of the normal breast tissue and the abnormal breast tissue according to the electromagnetic wave at 10 GHz have a bigger difference. In other words, when the electromagnetic wave at 10 GHz is transmitted through the breast tissue of the human body, the attenuation quantity which is generated by the electromagnetic wave in response to the normal breast tissue is distinctively different from the attenuation quantity which is generated by the electromagnetic wave in response to the abnormal breast tissue. Therefore, in one embodiment, the biological tissues which are scanned by the scan circuit 110 include, for example, the breast tissue, and the scan band corresponding to the breast tissue can be, for example, 10 GHz. As a result, the microwave imaging device 100 can scan to find out the normal breast tissue and the abnormal breast tumor by using the electromagnetic wave at 10 GHz.

In addition, considering the nerve tissue of the human body, the conductivity characteristics and the dielectric constants of the normal nerve tissue and the abnormal nerve tissue according to the electromagnetic wave at 26 GHz have a bigger difference. Therefore, in one embodiment, the biological tissues which are scanned by the scan circuit 110 include, for example, the nerve tissue, and the scan band corresponding to the nerve tissue can be, for example, 26 GHz. In other words, the microwave imaging device 100 can scan to find out the normal nerve tissue and the abnonnal nerve tissue by using the electromagnetic wave at 26 GHz.

Considering the energy values which are generated by the reception circuit 120, as shown in Step S240, the image generator 130 can look up a plurality of gray level look-up tables by using the energy values to generate a plurality of gray level values, and generates a detection image corresponding to the biological tissues according to the gray level values. To be more specific, as shown in Step S241, the computing unit 131 can select the gray level look-up tables according to the scan bands which are covered by the electromagnetic waves. For example, the computing unit 131 can select the gray level look-up table corresponding to the breast tissue according to the 10 GHz scan band, and the computing unit 131 can select the gray level look-up table corresponding to the nerve tissue according to the 26 GHz scan band.

As shown in Step S242 and Step S243, the computing unit 131 can calculate a plurality of attenuation quantities according to the energy values, and can look up the gray level look-up tables according to the calculated attenuation quantities, so as to generate the gray level values. In addition, as shown in Step S244, the drawing unit 132 can generate the detection image corresponding to the biological tissues according to the gray level values. For example, the breast tissue of the human body is a specific biological tissue in the biological tissues, and the specific biological tissue is corresponding to a specific scan band (such as, 10 GHz) in the scan bands. The scan circuit 110 can scan the specific biological tissue (such as, breast tissue) in the human body through the electromagnetic wave at the specific scan band (such as, 10 GHz). The computing unit 131 can calculate the attenuation quantity of the electromagnetic wave transmitted through the human body according to the energy value of the electromagnetic wave at the specific scan band (such as, 10 GHz). The pixels having the first gray level value in the detection image are used to represent the healthy tissue (such as, normal breast tissue), and the pixels having the second gray level value are used to represent the malignant tissue (such as, abnormal breast tissue).

In summary, in the invention, the electromagnetic waves are used to scan the target object, the gray level values of the detection image are generated according to the energy values of the electromagnetic waves which pass through the target body, and the attenuation quantities of the electromagnetic waves are simultaneously used to comprehend the electrical properties of the biological tissues of the related target body. Therefore, the cost of the hardware of the microwave imaging device is reduced so as to reduce the detection fee effectively when the microwave imaging device is applied to clinical diagnosis so that the burden of the patient is effectively reduced.

It will be apparent to those skilled in the art that various modifications and variations can be made to the structure of the invention without detaching from the scope or spirit of the invention.

What is claimed is:

1. A microwave imaging device, comprising:
a scan circuit, transmitting a plurality of electromagnetic waves in a plurality of scan bands toward a target object, wherein the scan bands are respectively corresponding to a plurality of biological tissues in the target object;
a reception circuit, receiving the electromagnetic waves passing through the target object, and generating a plurality of energy values according to the received electromagnetic waves; and
an image generator, looking up a plurality of gray level look-up tables by using the energy values to generate a plurality of gray level values, and generating a detection image corresponding to the biological tissues according to the gray level values.

2. The microwave imaging device as recited in claim 1, wherein the scan circuit comprises:
a transmitting antenna array; and
a transmitter, transmitting a plurality of driving signals to the transmitting antenna array, such that the transmitting antenna array transmits the electromagnetic waves.

3. The microwave imaging device as recited in claim 2, wherein the reception circuit comprises:
a receiving antenna array, receiving the electromagnetic waves passing through the target object; and
a receiver, calculating the energy values according to the electromagnetic waves received by the receiving antenna array.

4. The microwave imaging device as recited in claim 3, wherein each of the transmitting antenna array and the receiving antenna array comprises a plurality of antenna elements to make the microwave imaging device have a multiple-input multiple-output transmission mechanism.

5. The microwave imaging device as recited in claim 1, wherein the image generator comprises:
a computing unit, calculating a plurality of attenuation quantities according to the energy values, and looking up the gray level look-up tables according to the attenuation quantities, so as to generate the gray level values; and
a drawing unit, generating the detection image according to the gray level values.

6. The microwave imaging device as recited in claim 5, wherein the computing unit selects the gray level look-up tables according to the scan bands covered by the electromagnetic waves.

7. The microwave imaging device as recited in claim 5, wherein the energy values respectively are a received signal-strength indicator value.

8. The microwave imaging device as recited in claim 1, wherein the target object is a human body, the biological tissues comprises a breast tissue, and the scan band corresponding to the breast tissue is 10 GHz.

9. The microwave imaging device as recited in claim 8, wherein the biological tissues further comprises a nerve tissue, and the scan band corresponding to the nerve tissue is 26 GHz.

10. A microwave imaging method, adapted to a microwave imaging device, and comprising:
transmitting a plurality of electromagnetic waves in a plurality of scan bands toward a target object through a transmitting antenna array in the microwave imaging device, wherein the scan bands are respectively corresponding to a plurality of biological tissues in the target object;
receiving the electromagnetic waves passing through the target object through a receiving antenna array in the microwave imaging device; and
generating a plurality of energy values according to the received electromagnetic waves; and
looking up a plurality of gray level look-up tables by using the energy values to generate a plurality of gray level values, and generating a detection image corresponding to the biological tissues according to the gray level values.

11. The microwave imaging method as recited in claim 10, wherein the step of looking up the gray level look-up tables by using the energy values to generate the gray level values, and generating the detection image corresponding to the biological tissues according to the gray level values further comprise:
calculating a plurality of attenuation quantities according to the energy values;
looking up the gray level look-up tables according to the attenuation quantities, so as to generate the gray level values; and
generating the detection image according to the gray level values.

12. The microwave imaging method as recited in claim 11, wherein the step of looking up the gray level look-up tables by using the energy values to generate the gray level values, and generating the detection image corresponding to the biological tissues according to the gray level values further comprise:
selecting the gray level look-up tables according to the scan bands covered by the electromagnetic waves.

13. The microwave imaging method as recited in claim 12, wherein the energy values respectively are a received signal-strength indicator value.

14. The microwave imaging method as recited in claim 10, further comprising:
transmitting a plurality of driving signals to the transmitting antenna array through a transmitter in the microwave imaging device to make the transmitting antenna array transmit the electromagnetic waves.

15. The microwave imaging method as recited in claim 10, further comprising:
calculating the energy values through a receiver in the microwave imaging device.

16. The microwave imaging method as recited in claim 10, wherein each of the transmitting antenna array and the receiving antenna array comprises a plurality of antenna elements to make the microwave imaging device have a multiple-input multiple-output transmission mechanism.

17. The microwave imaging method as recited in claim 10, wherein the target object is a human body, the biological tissues comprises a breast tissue, and the scan band corresponding to the breast tissue is 10 GHz.

18. The microwave imaging method as recited in claim 17, wherein the biological tissues comprises a nerve tissue, and the scan band corresponding to the nerve tissue is 26 GHz.
